(19) United States
(12) Patent Application Publication
(10) Pub. No.: US 2012/0017547 A1
(43) Pub. Date: Jan. 26, 2012

(54) Measuring device and method for determining a surface contour on a human or animal

(76) Inventor: Christoph Rieser, Obersteinebach (DE)

(21) Appl. No.: 13/256,001

(22) PCT Filed: Mar. 12, 2010
(86) PCT No.: PCT/EP10/53198

§ 371 Date: Oct. 13, 2011

(30) Foreign Application Priority Data
Mar. 12, 2009 (DE) 102009012467.5
Apr. 14, 2009 (DE) 102009016970.9

(51) Int. Cl. B68F 3/00; A61B 5/107; G01B 1/00

(52) U.S. Cl. 54/44.1; 33/511; 33/512

(57) ABSTRACT

The invention relates to a measuring device (1) for determining a surface contour (42) of a human or animal body (41), wherein the measuring device (1) comprises a main chain (43) having several main-chain links (44, 44′, 44″, 44′″, 44^{lv}, 44^{v}), which are connected to each other by a swivel joint (45), characterized in that the swivel joint (45) has a scale (13, 23, 74′) or sensors, by means of which the angle between the at least two main-chain links (44, 44′, 44″, 44′″, 44^{lv}, 44^{v}) can be read or detected.

The invention relates to a measuring device for determining a surface contour on a human or animal comprising several chain links interconnected in a hinged manner.

Such measuring devices are known. For example, an articulated limit gauge for determining the dimensions and profiles of animal bodies is known from document DE 100 464 A, the limit gauge comprising two multi-link, articulated straps which extend towards the right and the left from a central body, and whose individual links latch into each other with wave-like raised portions and are thus fixed in the predetermined direction. This limit gauge is disadvantageous in that, on the one hand, it is only possible to measure a single cross section of the animal body because only the circumference of the animal body in a single dimension is measured by means of the limit gauge. Thus, a measurement of a three-dimensional surface cannot be realized, or only by comparatively frequent application of the limit gauge and the inaccuracies resulting therefrom. On the other hand, the links of the limit gauge have no scale units that would permit a numerical determination of the angular relationships between the individual links. As a consequence, the surface contour cannot be determined by numerical parameters. Thus, the measured surface contour is also not readily reproducible if the limit gauge has been readjusted.

It is the object of the present invention to provide a measuring device of the type mentioned in the introduction which does not exhibit the drawbacks of the prior art, and which, furthermore, is easy to handle, comparatively inexpensively to produce and, in particular, permits a reliable reproduction of the surface.

According to the invention, this object is accomplished by a measuring device for determining a surface contour on a human or animal according to claim 1, as well as by a method and a use according to the independent claims. Advantageous embodiments and developments of the present invention are apparent from the dependent claims as well as from the description with reference to the drawings. It must be remarked that the features cited individually in the patent claims can be combined in any technologically meaningful manner and depict other embodiments of the invention. The description, in particular in connection with the figures, additionally characterizes and specifies the invention.

The measuring device according to the invention comprises a main chain with a plurality of main-chain links which are interconnected by a hinge joint. The device is characterized in that a scale means or sensors, with which the angle between two chain links can respectively be read off or determined, are provided on the hinge joints.

The term scale or scale means within the sense of the invention must be interpreted broadly and also includes a vernier, for example. The measuring device according to the invention senses and in particular numerically evaluates a surface contour, so that the surface contour can advantageously be documented, compared and/or reproduced in a simple manner despite a potentially complex contour. Generally, the measuring device is adapted to the surface by means of a simple positive engagement between the measuring device and the surface, which is at least approximated to the surface, so that the measuring device can easily be operated even by a layperson. The modular construction from individual chain links moreover enables a comparatively versatile and precise adaptation of the measuring device to surface contours of different sizes and different shapes. The construction from preferably individual identical or at least similar chain links moreover permits a comparatively inexpensive production of the measuring device.

Advantageously, a numerical evaluation of the relative positions of two adjacent chain links is enabled by providing the scales. This offers the advantage that, after the adaptation of the measuring device to the surface contour to be measured, the position of each of the chain links of the measuring device is uniquely determined in a numerical, documentable and reproducible way. Moreover, it is thus possible to carry out a remote transmission of the measured values, for example a transmission in an acoustical way, in particular by means of language, or electronically. In order to uniquely document the entire surface contour, it is therefore sufficient to note down the appropriate scale values of the respective scales after the measuring device has been adapted. In this way, it is possible, for example, to compare measured scale values with previous scale values, whereby the change of the surface, for example a horse's back, can be determined, for instance, which may be of orthopedic interest. Moreover, the reproduction and mechanical replication of the surface contour at a location remote from the measuring site is possible. For example, the scale values can be communicated in writing of by telephone to a saddlery, which can then reproduce the contour of the horse's back on-site, so that a saddle can be manufactured by the saddlery that is optimally adapted to the contour of the horse's back without a saddler having to take measure of the horse personally. This is aided by the fact that the measuring device comprised of the chain links according to the invention, in contrast to electronic methods for determining a horse's back, is comparatively simple to operate by the respective owner of the horse himself.

The property that the measuring device can be folded together due to the plurality of hinge joint makes it possible to store the measuring device in a space-saving manner, to transport it easily and to simply send it by post between the saddlery and the owner of the horse. On the other hand, it is conceivable, due to the low production costs, that businesses working with horses are equipped, for example, with the measuring device on a large-scale basis.

In an alternative embodiment of the present invention, it is conceivable that the hinge joints are equipped with sensors, so that the relative angular position, that is, associated “scale values”, are read out electronically and are transmitted to an evaluation and/or display unit, preferably wirelessly.

According to a preferred embodiment, it is provided that the measuring device comprises at least one, preferably more, side chains, which make a three-dimensional acquisition of the surface possible. For example, the side chains, if oriented in a straight line, are disposed substantially parallel to one another. In this way, the curvature of the ribs can be measured in different areas of the horse's back by means of the plurality of side chains. In this case, the side chains are in particular disposed on both sides of the main chain, with the side chains being formed on both sides, preferably in mirror symmetry to one another in relation to the main chain.

The side chain is preferably attached to a chain link of the main chain, which link has two first end portions disposed in opposed relation along a first direction (which is hereinafter also referred to as primary direction), each of which comprises a first joint fitting, and wherein at least one of the chain links further comprises at least one second end portion disposed at an angle to the first direction and pointing in a secondary direction, which comprises a second joint fitting on which at least one further chain link of the side chain is provided.

Advantageously, the main-chain links and/or the side-chain links are at least partially constructionally identical so that a measuring device for mapping a larger surface contour can be realized by the connection of a plurality of constructionally identical chain links alone. In this case, such a measuring device can advantageously be adapted in a versatile manner and can be produced comparatively inexpensively. Thus, the measuring device can be produced from the constructionally identical chain link in a modular manner, because the size of the measuring device can be adjusted and adapted to the surface contour to be measured in the desired manner by simply adding or removing further chain links.

The main and/or side-chain links comprise, for example, tabs which are preferably produced from cardboard, wood, plastic or metal, in particular aluminum.

According to a preferred embodiment, it is provided that the main chain comprises a chain link to which two or more side chains are attached. Thus, the chain link is preferably configured in a cross shape. Advantageously, such a configuration of the device, which is symmetrical in relation to the main chain, enables the sensing of a back, in particular a horse's back, which is symmetrical in relation to the contour of the vertebrae, with the main chain following the contour of the spine, and the curvature of the ribs of the horse's back, which extends substantially perpendicularly to the spine on both sides of the spine, being sensed by means of the side chains.

According to a preferred development, it is provided that the first joint fittings comprise first bore holes parallel to the second direction and the second joint fittings comprise second bore holes parallel to the first direction. Advantageously, two chain links that are adjacent along the first or second direction can thus be connected in a simple manner by a joint bolt being disposed in the corresponding first or second bore holes. As an alternative, it is conceivable to form at least one of the first or second bore holes by equivalent first and second pivoting axes and to push the other one of the first and second bore hole of an adjacent chain link onto the first or second pivoting axis. Instead of the first or second bore hole, a chain link having first or second clips is in this case also conceivable, which are clipped onto the first or second pivoting axis. It is a matter of course for a person skilled in the art that any other known option of an articulated connection of two adjacent chain links is conceivable so long as the two adjacent chain links are interconnected in a hinged manner. The joint bolt comprises, for example, a screw, a clamp and/or a rivet. In an alternative embodiment of the chain link and the further chain link consisting of cardboard, it is conceivable that the joint bolt comprises a head clamp or split pin.

According to a preferred development, it is provided that at least a first joint fitting and/or a second joint fitting each comprise a scale with scale divisions, with the scale divisions of the first joint fitting being disposed substantially parallel to the circumference of the first bore hole and the scale divisions of the second joint fitting being disposed substantially parallel to the circumference of the second bore hole. Thus, the angle between two adjacent chain links can advantageously be read off directly on the scale. In a preferred embodiment, one of the chain links in this case comprises a joint fitting with a scale having only a single scale line, whereas the further joint fitting of the adjacent other chain link which is connected to the joint fitting in a hinged manner has a scale with a plurality of scale lines that are numbered with scale values. Thus, the one scale line of the chain link marks a particular scale value on the scale of the further chain link, which depends on the angle between the chain link and the further chain link.

In an alternative, particularly simple embodiment, it is conceivable that the scale on the joint fitting and on the further joint fitting connected with the joint fitting respectively only has a single scale division formed by a marker line extending over both joint fittings. This marker line is preferably produced by means of a pen by the user of the measuring device after adaptation to the surface to be measured, with the angular position between the joint fitting and the further joint fitting being uniquely determined by the marker line. In this case, the measuring device is preferably made of cardboard, wood and/or plastic and is provided as a disposable or reusable measuring device that is comparatively inexpensive to produce and particularly convenient to send. By means of the marker line, a reconstruction of the surface to be measured is possible, which is then transferred, in particular at the saddlery, onto a further measuring device, for example, which is made of metal or plastic and comprises a plurality of numerical scale values. The marker line preferably comprises a partial line on the joint fitting and another partial line on the further joint fitting, which substantially come into alignment with each other in the predetermined angular position.

According to a preferred development, it is provided that the first joint fittings along the second direction and the second joint fittings along the first direction are disposed offset relative to one another, so that advantageously a chain is formed in a straight manner relative to the first or second direction from a plurality of chain links and further chain links adjacent along the first or second direction.

According to a preferred development, it is provided that the distance between the first joint fittings is unequal to the distance between the second joint fittings. Advantageously, the chain link can thus be universally adapted to the surface contour of a human or animal. For example, the distances between the first joint fittings, i.e. along the first direction, are greater than the distances along the second direction in order to measure a spine along the first direction and a curvature of the ribs of a horse along the second direction. This is advantageous particularly because the curvature of the spine is naturally significantly less pronounced than the curvature of the ribs. Particularly preferably, the distance between the first joint fittings substantially corresponds to a mean vertebral length of the spine of the horse in order to obtain a reproduction of the spine that is as true to nature as possible. Alternatively, it is also conceivable that, along the first or second direction, different chain links have different distances between the first or the second hinge joints.

In another advantageous embodiment, a mark which uniquely identifies each chain link, for example a number, is provided on each chain link of the main and/or the side chain in order to facilitate the transmission of the scale values.

In a preferred embodiment of the present invention, the measuring device comprises at least one means for determining at least one direction of inclination of a chain link. The position of the sensed surface is defined in space in a reproducible manner by means of this “reference orientation” of one or more chain links. This information may be important for example in order to analyze the position of the sensed surface in space. This information is particularly important in the production of saddles, for example in order to determine the seat position of the rider and to then determine the dimensioning, design or arrangement of the saddle flaps, pommels and stirrup holders by means of this information. Preferably, the means for determining at least one direction of inclination is a bubble level with a scale means. Such a means can be provided on a chain link of the primary and/or secondary direction. Preferably, the bubble level is provided on a chain link of the primary direction. Preferably, it is a level pivotable relative to the chain link disposed in such a way that the inclination of the chain link with regard to its longitudinal orientation is measured relative to the field of gravity. Even more preferably, the level is directly attached to at least one chain link adjacent to the center of the main chain.

According to a preferred development, it is provided that the hinge joints of the main-chain links and/or side-chain links comprise latching means, for example positive-engagement means. For example, the first joint fittings comprise a first furrowed portion and/or the second joint fittings comprise a second furrowed portion. Advantageously, a fixation of the angle between two adjacent chain links is thus obtained so that an inadvertent readjustment of the measured angle is prevented. To this end, two adjacent furrowed portions preferably engage each other, so that a latching of the positive-engagement means in the respective angular position, for example, is obtained. The latching improves safety during operation of the measuring device because only defined orientations are made possible and the errors that result from an erroneous read-off and which might possibly “be magnified” with an increasing chain length are prevented.

In this case, the positive-engagement means are preferably pressed onto each other by means of a biasing element, so that a readjustment of the angular position is possible only if the biasing force exerted by the biasing element on the positive-engagement means is overcome.

In a preferred embodiment, the first furrowed portion in this case is respectively disposed in the radial direction relative to the first bore hole, and the second furrowed portion is respectively disposed in the radial direction relative to the second bore hole. Particularly preferably, the first and second furrowed portion comprises a distance of the individual furrows corresponding to the distance of the scale divisions of the first and second scales, so that reading off the scale divisions is made significantly easier, because in this case, an adjustment of the angle between the scale divisions is not possible.

According to a preferred development, it is provided that the hinge joint comprises friction-engagement means. Advantageously, the force required for the relative adjustment of the two joint fittings during the cooperation of two adjacent joint fittings pivoted to one another is prevented by the friction means, so that an inadvertent readjustment of the angle is prevented. To this end, the friction-engagement means preferably comprises a contact surface, which is disposed in particular concentrically about the bore hole and is pressed onto a complementary further contact surface, in particular by means of the screw, clamp, rivet and/or spring, so that a frictional engagement is generated between the contact surface and the further contact surface. In this case, the chain links are preferably made of cardboard. Preferably, the joint bolt comprises a spring for generating a force-fitting and/or positive connection between the first furrowed portion and a further furrowed portion of the further joint fitting. Advantageously, the furrowed portion of a chain link and the corresponding furrowed portion of an adjacent chain link are pressed together by the spring pressure originating from the spring, so that the angle between the chain link and the adjacent chain link can only be adjusted if the spring force is overcome. The chain link and the adjacent further chain link are fixed relative to each other by the mutual latching of the furrowed portion. An inadvertent readjustment of the angle is thus prevented. It is conceivable that friction is effected magnetically or electro-magnetically.

According to a preferred embodiment, locking means are provided which are able to fix the hinge joints of the measuring device according to the invention in their respective angular position and, for example, lock the positive-engagement means in their positive position. For example, at least one displaceable locking bar is provided which locks the positive-engagement means in their latched position. For example, the locking bar is driven into the locking position by a rotatable cam, with its return into the unlocked position being accomplished by means of an elastic bias by a spring.

The “stiffening capability” of the measuring device due to the friction-engagement means or the positive-engagement means, more preferably in combination with locking means, is advantageous in that the measuring device can be used not only for taking measures but also for checking measures, for example by a surface contour being checked for deviations with a pre-set measuring device. For example, a finished saddle is thus checked by means of a measuring device which was set to the scale values previously taken from a horse and “stiffened” by means of friction-engagement, positive-engagement means and/or locking means.

According to another preferred embodiment, it is provided that the main-chain link to which the side chain is attached enables a pivoting adjustment of the side chain relative to the main chain. In a particularly preferred embodiment, the pivoting adjustment means comprises other positive and/or friction-engagement means for latching and/or fixing the respective pivot position and preferably a scale whereby the respective pivot position is numerically determined and reproducible.

According to a preferred development, it is provided that the distance between two further end portions of at least one chain link can be adjusted by means of a longitudinal adjustment means, wherein the longitudinal adjustment means preferably comprises an elongated hole and a further scale disposed along the elongated hole. Preferably, the longitudinal adjustment means also comprises positive and/or friction-engagement means, in particular a furrowed portion and/or contact surface, so that an inadvertent longitudinal adjustment is prevented.

Another subject matter of the present invention is a method for determining a surface contour of a human or animal body, preferably of a horse's back, with a measuring device according to the invention, wherein, in the first method step, the measuring device is disposed on the surface and the main links and, if applicable, the side links are adapted to the surface contour, and wherein, in a second method step, scale values of the measuring device are read off.

In a preferred embodiment of the method, at least one direction of inclination of a main-chain link or side-chain link, preferably of a main-chain link, is determined in the second method step. Advantageously, the surface contour, i.e. the horse's back, is thus uniquely numerically determined and is freely reproducible at any time. In particular, a comparison of the measured scale values with previous scale values is possible.

Another subject matter of the present invention is a use of the measuring device according to the invention for the manufacture, adaptation and/or checking of equestrian equipment, in particular a saddle. Advantageously, the horse's back is thus measured in its entirety in an easily handled manner, with the surface contour of the horse's back becoming numerically documentable in the form of the scale values. This makes it possible to produce and/or adapt the equestrian equipment solely on the basis of the scale values. In the process, the scale values are preferably used as input parameters for a CNC machine so that the equestrian equipment can be produced automatically and, at the same time, is individually adapted to the horse concerned. Particularly, this makes it possible to order, for example electronically and/or by telephone, individualized equestrian equipment, with the horse's owner himself simply determining the appropriate production parameters, so that considerable cost can be saved. In the control step provided according to the invention, a check of equestrian equipment by means of the measuring device is provided, wherein, for example in the third method step, the measuring device or a measuring device newly adjusted in accordance with the scale values is inserted into an equestrian equipment, e.g. a saddle, which already exits, so that any deviation between the equestrian equipment and the surface contour of the horse's back can be analyzed with reference to a deviation between the equestrian equipment and the measuring device.

For example, the measuring device is used for individually adapting the saddle contact surface of a saddle rack to a horse's back which was previously determined by the measuring device. A saddle rack serves for testing an individually produced saddle. For example, the saddle rack is produced in accordance with the scale values determined by the measuring device, or comprises an adjustment option for adapting the saddle contact surface.

Exemplary embodiments of the invention are shown in the drawings and explained in more detail in the description below. In the drawings:

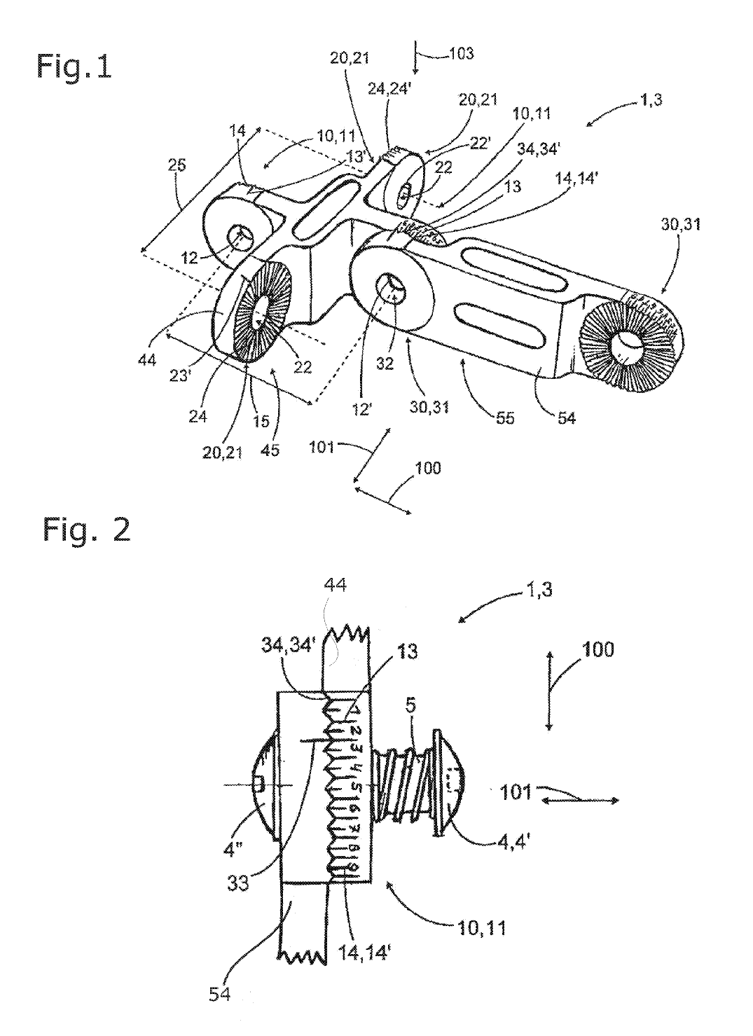

FIG. 1 shows a schematic perspective view of two chain links, i.e. a main-chain link 44 according to a first embodiment and a side-chain link 54 according to a first embodiment;

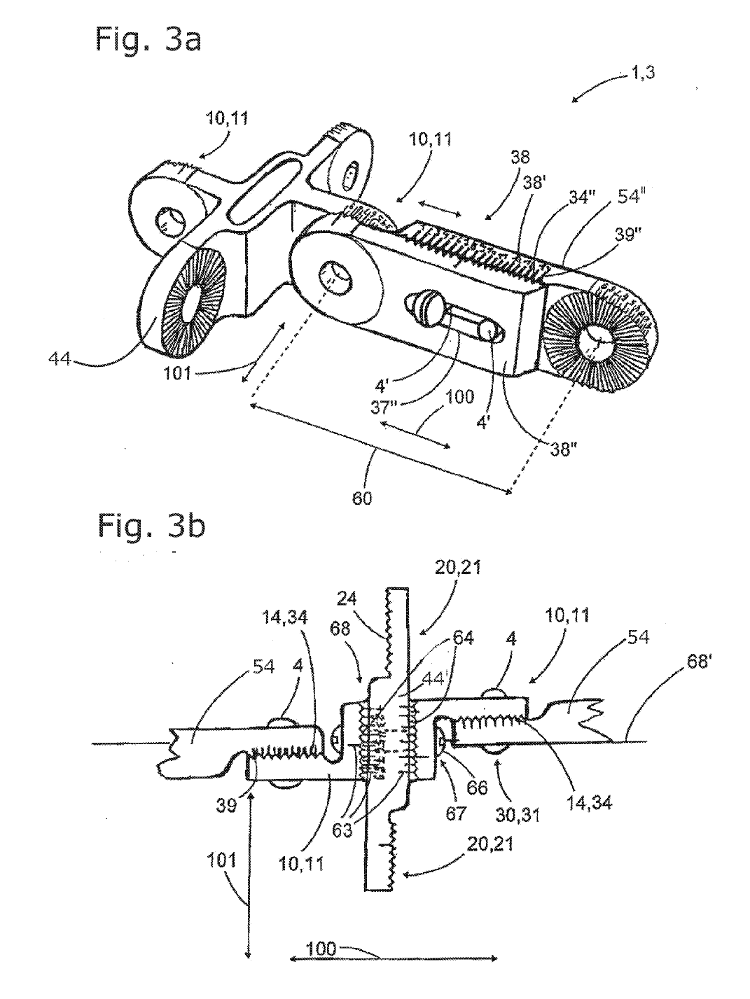

FIG. 2 is a detailed view of the hinge joint between the main-chain link 44 and the side-chain link from FIG. 1;

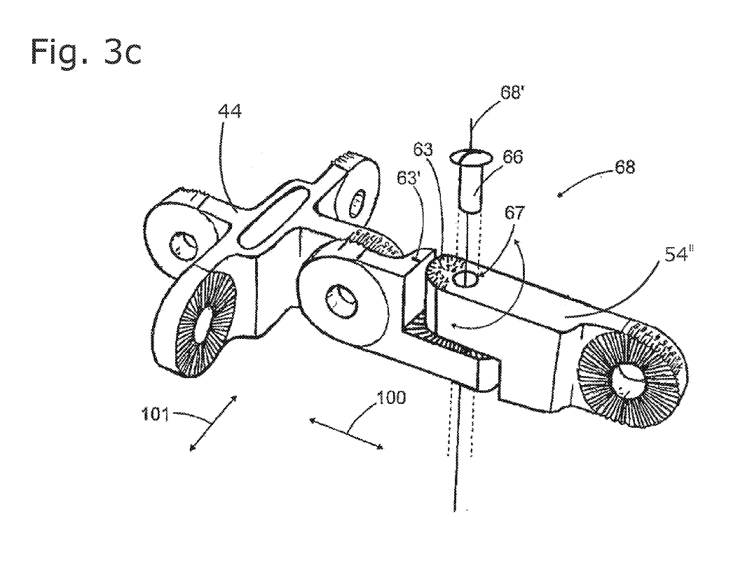

FIG. 3a shows a schematic perspective view of two chain links, i.e. the main-chain link 44 according to the first embodiment and a side-chain link 54′ according to a second embodiment;

FIG. 3b shows a schematic top view of two chain links, i.e. the main-chain link 44′ according to a second embodiment and a side-chain link 54 according to the first embodiment;

FIG. 3c shows a schematic perspective view of two chain links, i.e. the main-chain link 44 according to the first embodiment and a side-chain link 54″ according to a third embodiment;

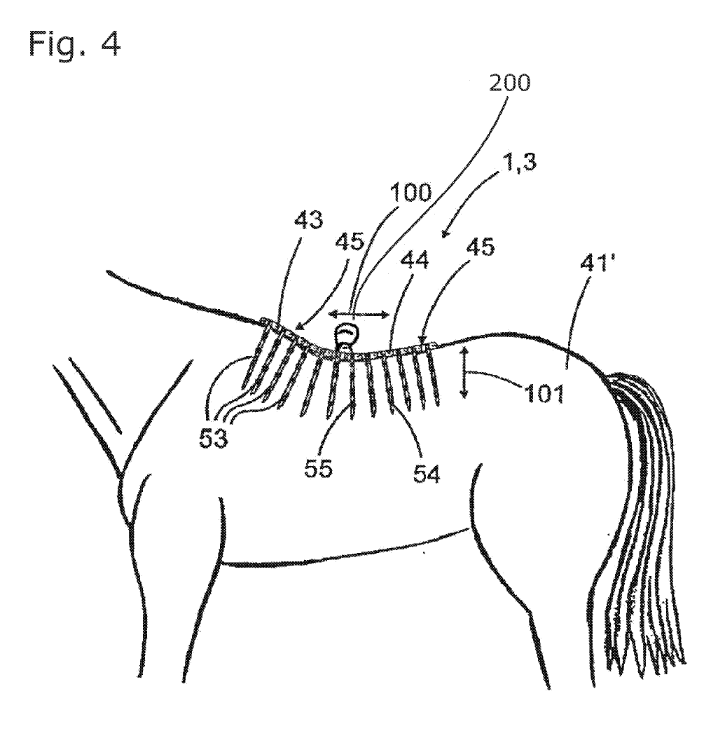

FIG. 4 shows a schematic view of a use of the measuring device according to the invention in taking measures on a horse's back;

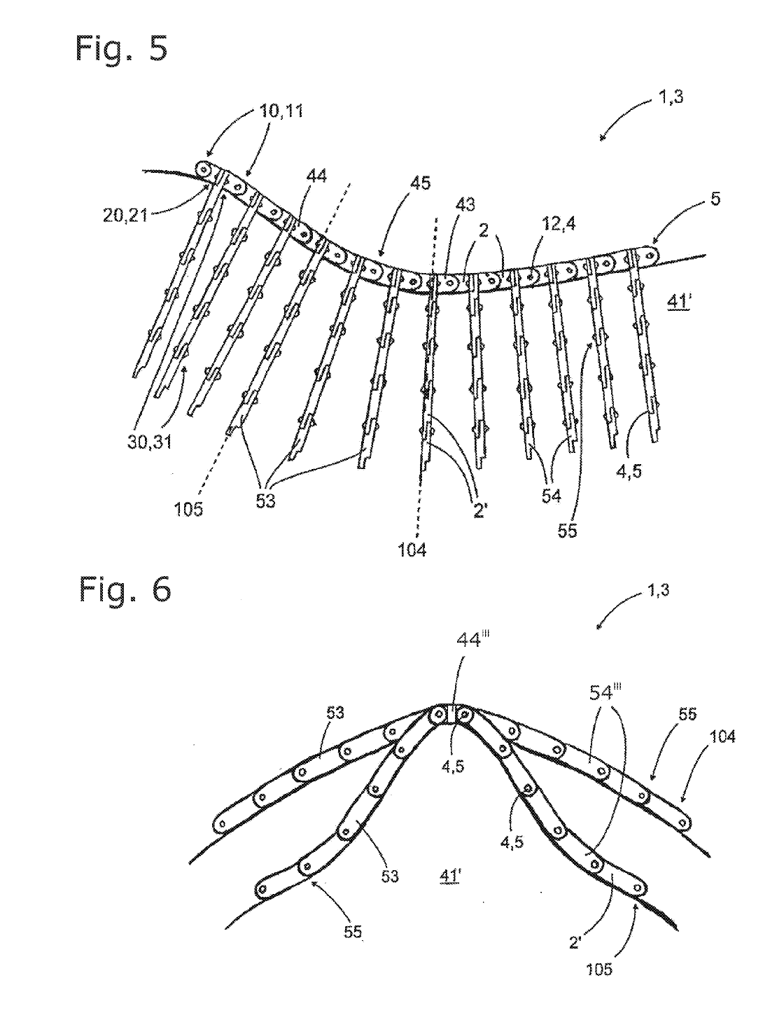

FIG. 5 shows a detailed view of FIG. 4;

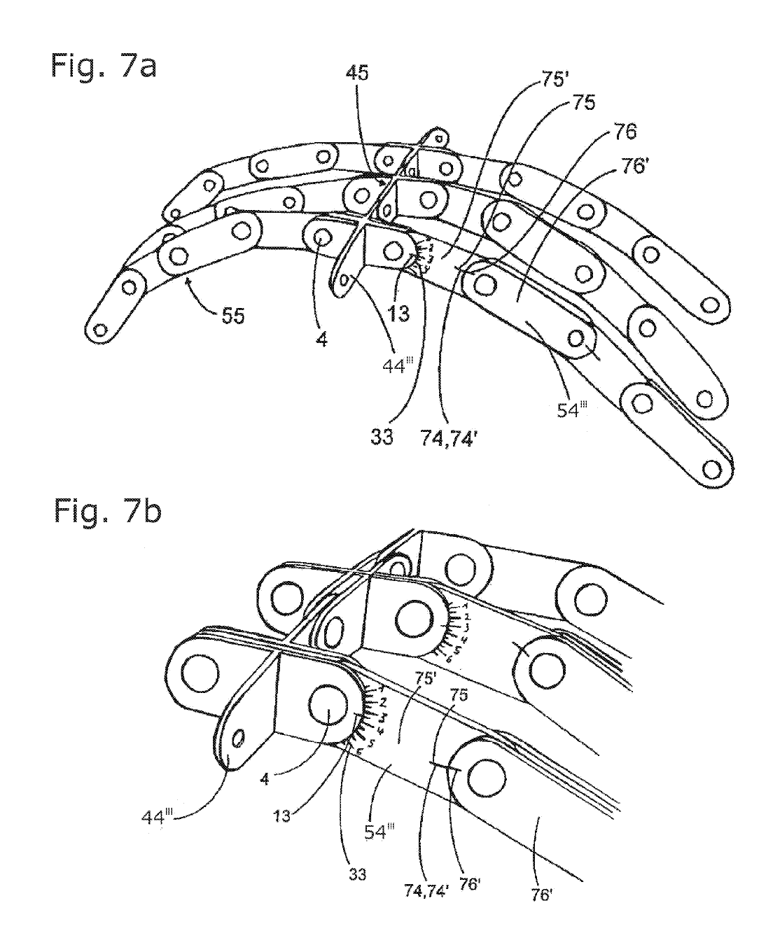

FIG. 6 shows a detailed view of the measuring device from FIG. 4 viewed in the direction of the contour of the spine;

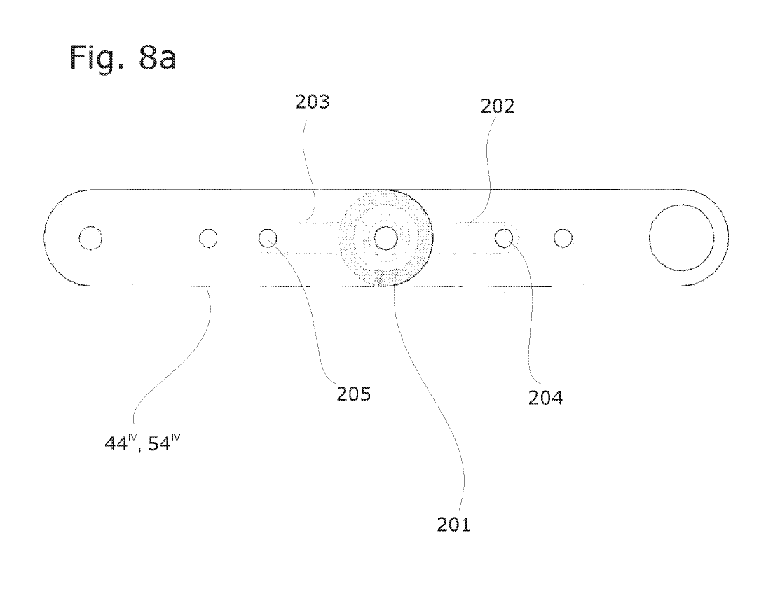

FIGS. 7a, 7b show schematic views of a measuring device according to a further embodiment of the present invention;

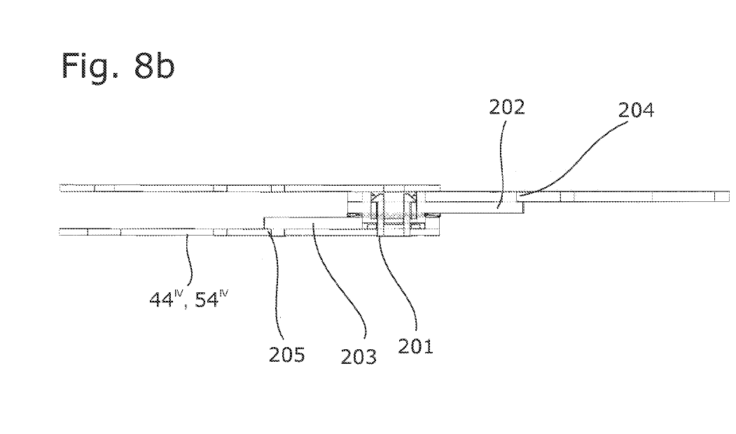

FIGS. 8a, 8b show schematic views of a measuring device according to still another embodiment of the present invention;

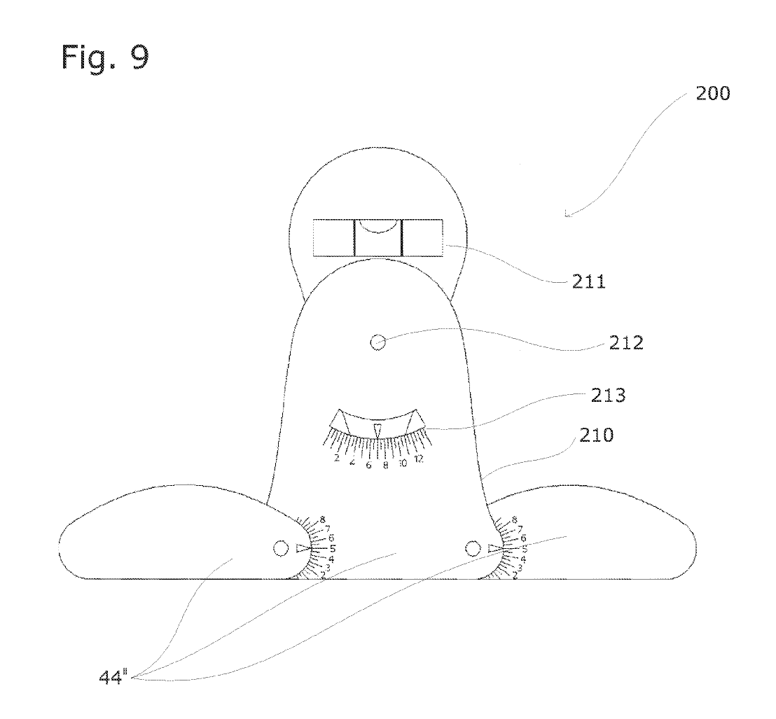

FIG. 9 shows a detailed view of the means 200 for determining a direction of inclination of a main-chain link;

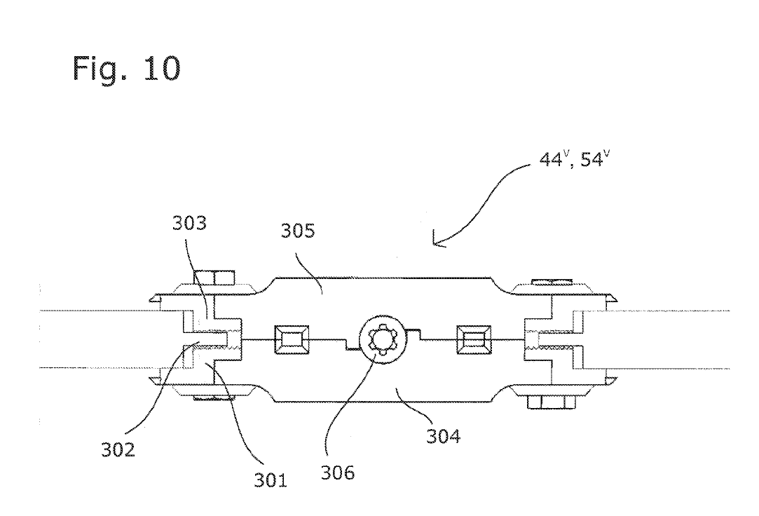

FIGS. 10 to 12 show another embodiment of the measuring device in which the main and/or side-chain links moreover comprise locking means.

FIG. 1 shows a schematic perspective view of a part of the measuring device 1 according to the invention in a first embodiment, with the chain link 44 of the main chain comprising a total of four joint fittings 11, 21, all of which lie in a main extension plane 102 of the chain link 44. In this case, two first joint fittings 11 are disposed on two opposing first end portions 10 of the chain link 44, whereas two second joint fittings 12 are disposed on two opposing second end portions 20. The first joint fittings 11 are substantially disposed along a first direction 100 and the second joint fittings 21 are substantially disposed along a second direction 101 which is perpendicular to the first direction 100. Thus, the chain link 44 has a type of cross-structure and can therefore be connected in a hinged manner, both along the first direction 100 at the two first end portions 10, as well as along the second direction 101 at the two end portions 20, to adjacent further main-chain links (not shown) or side-chain links 54, respectively. To this end, the first joint fittings 11 respectively comprise a first bore hole 12 and a first positive-engagement means 14 in the form 10 of a first furrowed portion 14′ and the second joint fittings 21 respectively comprise a second bore hole 22 and a second positive-engagement means 24 in the form of a second furrowed portion 24′. Furthermore, one of the first joint fitting 11 respectively comprises a scale 13 with a plurality of scale divisions disposed along the circumference of the corresponding bore hole 12, with the other of the two first 15 joint fittings 11 comprising a scale 13 with precisely one scale line 13′. Analogously, the two second joint fittings 21 are equipped with two second scales 23. By way of example, only one adjacent side-chain link 54 is shown for the sake of clarity, which is connected in a hinged manner to the main-chain link 44 by means of a cooperation of a first joint fitting 11 with a further joint fitting 31 of this chain link 54. In this case, the first bore hole 12 is disposed so as to be congruent with another bore hole 32 of the further joint fitting 32, with both bore holes 12, 32 being oriented parallel to the second direction 101 and the first and the further joint fitting 11, 31 being held together by a joint bolt 3, which is not shown and is disposed in the first and the further bore 12, 32, in such a way that the first furrowed portion 14′ engages a further furrowed portion 34′ of a complementary further positive-engagement means 34 of the further joint fitting 31. The further joint fitting 31 moreover comprises a further scale 33 with a scale line 33′ disposed adjacent to the plurality of 30 scale divisions of the first scale 13, so that the scale line 33′ of the further scale 33 marks a scale division of the first scale 13 depending on the angle between the main-chain link 44 and the side-chain link 54 with regard to a rotation about the joint bolt 3. Thus, the associated scale value is a unique and reproducible measure for the relative position between the main-chain link 44 and the side-chain link 54. The first and further furrowed portions 14′, 34′ cause a latching between the main-chain link 44 and the side-chain link 54 in the respective angular position. The distances between the individual latching positions in this case always substantially correspond to the distances between the 5 individual scale divisions, so that exactly one scale value of the scale 13 always corresponds to a latching position. Reference is made to the fact that the hinged connection of the chain links is mutually compatible. Preferably, several main-chain links 44 are connected in a hinged manner in the direction 101 in the orientation shown in FIG. 1 in order to form a main chain, and several side-chain links 54 are connected in the direction 100 in order to form the side chain of the measuring device.

In FIG. 2, a lateral view from the direction of the arrow 103 shown in FIG. 1 is shown, with the joint bolt 4 being additionally depicted in FIG. 2. The joint bolt 4 comprises a screw 4′ and a sleeve 4″ comprising an internal thread, into which sleeve the screw 4′ is screwed so that the first joint fitting 11 and the further joint fitting 31 are being held together in a hinged manner by the heads of the screw 4′ and of the sleeve 4″. Furthermore, the joint bolt 4 comprises a spring 5 which causes the first furrowed portion 14′ and the second furrowed portion 34′ to engage so that the spring force of the spring 5 has to be overcome for adjusting the angle between the chain link 2 and the further chain link 2′ so that the first and the further furrowed portions 14, 34 can be brought out of engagement.

FIG. 3a shows a schematic perspective view of a chain link according to the first embodiment of the present invention and of a assembly 3 of a chain link according to a second embodiment of the present invention, with FIG. 3 being substantially identical to FIG. 1, with the side-chain link 54′ centrally having a longitudinal adjustment means 38 along the first direction 100. For this purpose, the chain link 54′ comprises a first section 38′ with first bore holes 37′ and a second section 38″ with an elongated hole 37″. The first and the second sections 38′ and 38″ are interconnected by means of two bolts 4′ which are fixedly disposed in the bore holes 37′ and are mounted so as to be longitudinally displaceable in the elongated hole 37″. The length 60 of the side-chain link 54′ can be adjusted along the first direction 100 due to the longitudinal displaceability of the bolts 4′. Furthermore, a first scale 39″ with a single scale line is disposed on the second section 38″, and a second scale 39″ with a plurality of scale divisions is disposed on the first section 38′, so that the relative position between the first section 38′ and the second section 38″ can be numerically determined. Furthermore, the first and the second sections 38′, 38″ comprise furrowed portions 34″ which prevent an inadvertent longitudinal readjustment and are held in mutual engagement by means of the springs disposed on the bolts 4′.

FIG. 3b shows a measuring device with a main-chain link 44′ according to a second embodiment of the present invention, wherein the chain link 44′ additionally comprises two pivoting adjustment means 68 for the side chains comprised of the side-chain links 54, wherein the first end portions 10, by means of the pivoting adjustment means 68, can be pivoted relative to the chain link 2 about a pivoting axis 68′ parallel to the first direction 100, in particular independently from each other. For this purpose, the chain link 44′ comprises a central bore hole 67 parallel to the first direction 100 in which a joint bolt 66 extends, to which the first end sections 10 with corresponding bore holes 67 are attached. Furthermore, the end sections 10 comprise positive-engagement means 64 and the rest of the chain link 2 comprises corresponding complementary positive-engagement means 64′ in the form of furrowed portions, so that the end sections 10 can be latched in relation to the rest of the chain link in their respective pivot positions. In order to specify numerically the respective pivot position, the pivoting adjustment means 68 each comprise scales 63 with corresponding scale divisions. Alternatively, it would also be conceivable to respectively link the second end portions 20, additionally or exclusively, to the side-chain links 54 by means of a pivoting adjustment means 68.

FIG. 3c shows another embodiment of the measuring device in which the side chain is linked to the main chain by means of a side-chain link 54″, which instead of the longitudinal adjustment means 38 comprises a pivoting adjustment means 68. For this purpose, the first section 38′ and the second section 38″ respectively comprise a congruent bore hole 67 for receiving a pivot bolt 66 defining the pivot axis 68′, wherein the first and second sections 38′, 38″ are pivotable relative to each other about the pivot axis 68′. In this case, the pivot axis 68′ substantially extends perpendicular both to the first as well as to the second direction 100, 101. In this case, the respective angular position between the first and the second section 38′, 38″ can be latched by means of a positive-engagement means 64, in particular in the form of furrowed portions 64′, so that an inadvertent pivoting is prevented. Furthermore, the first section 38′ comprises a scale 63 with a plurality of numbered scale divisions 10, whereas the second section 33″ is provided with a scale 63′ having a single scale line for marking a corresponding scale division depending on the respective angular position between the first and the second sections 38′, 38″ relative to the pivot axis 68′. Alternatively, it is also conceivable that the adjacent cross-like main-chain link 44 is equipped with such a pivoting adjustment means 68 for pivoting 15 at least one of the first and/or second end portions 10, 20.

FIGS. 4, 5 and 6 show schematic views of a measuring device 1 according to an embodiment of the present invention, wherein the measuring device 1 is disposed, by way of example, on a horse's back 20, 41′ in FIGS. 4, 5 and 6. The measuring device 1 in this case comprises a main chain 43 which substantially extends along a first direction 100 and is composed of a plurality of main-chain links 44. The main-chain links 44 in this case comprise, in particular, a plurality of the chain links 2 according to the invention shown in FIGS. 1, 2 and 3, which are respectively connected in a hinged manner by means of joint bolts 4. Furthermore, the measuring device 1 comprises a plurality of side chains 53, with each side chain substantially extending along a second direction 101 perpendicular to the first direction 100 and each side chain 53 being comprised of a plurality of side-chain links 54. The side-chain links 54 each comprise, in particular, a plurality of the further chain links 2′ shown in FIG. 1, which are partially connected in a hinged manner by means of joint bolts 4. The main chain 43 is disposed substantially along a spine of the horse's back 41′, so that the contour of the spine is reproduced by the relative positions of the adjacent main-chain links 44 relative to one another, whereas the side chains 53 disposed next to one another along the main chain 43, 21 reproduce the respective rib curvatures of the horse's back 41′ along the spine. As in the arrangement of chain links according to the FIGS. 1 and 2, each of the hinge joints 45, 55 comprises, between adjacent main-chain links 44, between adjacent 5 side-chain links 54 and between adjacent main and side-chain links 44, 54, a latching by means of corresponding positive-engagement means 14, 24, 34 and a scale 13, 33 which specifies numerically the angle between the respective adjacent elements. After pressing or positively adapting the measuring device 1 to the horse's back 10, 41′ in a first step, the scale values are to be read off in a second step from the scales 13, 33 of the different hinge joints 45, 55. The three-dimensional surface of the horse's back 41′ is uniquely and reproducibly characterized solely by the specification of these scale values and an association of these scale values with the corresponding hinge joints 45, 55. Moreover, a bubble level device 200 is provided for determining a direction of inclination of a central main-chain link 45. Since the position of the main-chain link 45 is defined by the contour of the spine of the horse's back, the determination of a direction of inclination of the main-chain link 45 is in this case sufficient in order to define the position of the measured surface in space. The saddle can be designed in accordance with this additionally obtained information, in particular with regard to the targeted seat position of the rider. The production and/or adaptation of equestrian equipment, such as a riding saddle, is thus possible based on the scale values alone.

FIG. 6 schematically shows a sectional view in accordance with the cut lines 104 and 105 illustrated in FIG. 5, with the reproduction of different rib curvatures in the region of the withers 104′ and in the region of the ribs 20, 105′ of the horse's back 41′ by means of different side chains 53 being illustrated by means of the sectional view. In a preferred embodiment, individual chain links 44, 54 are equipped with a pivoting adjustment means 68 so that contact of two adjacent side chains 53, for example in the case of a correspondingly strongly curved spine, can be prevented by pivoting the side chains 53 accordingly. In another preferred embodiment, it is conceivable that the linkage of each side chain 53 to a main-chain link 44 is respectively equipped with a pivoting adjustment means 38 so that all the side chains 53 can be aligned parallel to the field of gravity. Such an alignment of the side chains 53 could preferably be accomplished by means of levels, 30 which are disposed on the individual side-chain links 54.

The FIGS. 7a and 7b show schematic perspective views of a measuring device 1 according to another embodiment of the present invention, with the second embodiment being substantially identical to the measuring device 1 according to the first embodiment illustrated in FIGS. 4, 5 and 6, but wherein the main-chain links 44′″ and the side-chain links 54′″ are substantially, and in particular their tabs, being made from cardboard 5. Adjacent chain links 44′″ and 54′″ are interconnected by means of joint bolts 4 in the form of rivets. In order to increase torsional rigidity, the chain links 2 and the further chain links 2′ are at least partially given a double-walled design. Furthermore, the hinge joints 45, 55, in contrast to the previously shown embodiment, comprise no positive-engagement means in this embodiment, but instead friction-engagement means 71, 72, 73, with contact surfaces of adjacent friction-engagement means 71, 73 being pressed or clamped onto each other by the rivets, so that in order to adjust the angle of the adjacent main and/or side-chain links 44′″, 54′″ the frictional engagement between the corresponding contact surfaces must be overcome, thus reducing the danger of inadvertent readjustment of the angular position. The main and side-chain links 44′″, 54′″ in part comprise scales 13, 33 in accordance with the first embodiment, wherein the angle between the corresponding adjacent main and side-chain links 44′″, 54′″ can be read off numerically. Alternatively, some main and side-chain links 44′″, 54′″ comprise a scale 74′ which 20 only comprises a single marker line 74 which is only produced on the measuring device 1 by the user of the measuring device 1, for example by means of a felt-tipped pen. This marker line 74 is drawn in the set or measured angular position over two adjacent hinge joints 75′, 76′ that are interconnected in a hinged manner, so that a first partial line 75 is produced on the one 25 hinge joint 75′ and a second partial line 76 is produced on the adjacent other hinge joint 76′, and the corresponding angular position between the adjacent hinge joints 75′, 76′ is reproducibly defined by the alignment of the two partial lines 75, 76.

The FIGS. 8a and 8b show another embodiment of the measuring device with chain links 44^{IV}, 45^{IV} of the main and side chains made substantially from cardboard, with additional positive-engagement means 202, 203 in the form of furrowed disks of plastic being provided in the hinge-joint connection of the chain links 44^{IV}, 45^{IV}, the furrowed surfaces of which are pressed onto each other by spring bias. For blocking rotation, these positive-engagement means 202, 203 are provided with a lateral arm which respectively reaches into a through-hole 204, 205 of the chain links 44^{IV}, 45^{IV} with a protrusion.

FIG. 9 shows in detail the means 200 for determining a direction of inclination of a main-chain link 44″, with the latter differing from the previously shown embodiments as regards their lateral contour. The means 200 for determining the direction of inclination comprises a bubble level which is attached to the tab 210 of the chain link 44″ so as to be pivotable about an axis of rotation 212. The extent of pivoting can be read off a scale 213. The procedure generally is such that, after the measuring device has been put on and adapted to the surface to be measured, the part with the bubble level is balanced out by pivoting and the associated scale value is read off the scale means 213. If a less accurate reading accuracy is required, the bubble level could also be connected rigidly to the chain link, and the scale means could be provided on the level itself. A level with which balancing is possible in all directions and with which the inclination of the chain link relative to at least two orthogonal directions can be determined may also be provided.

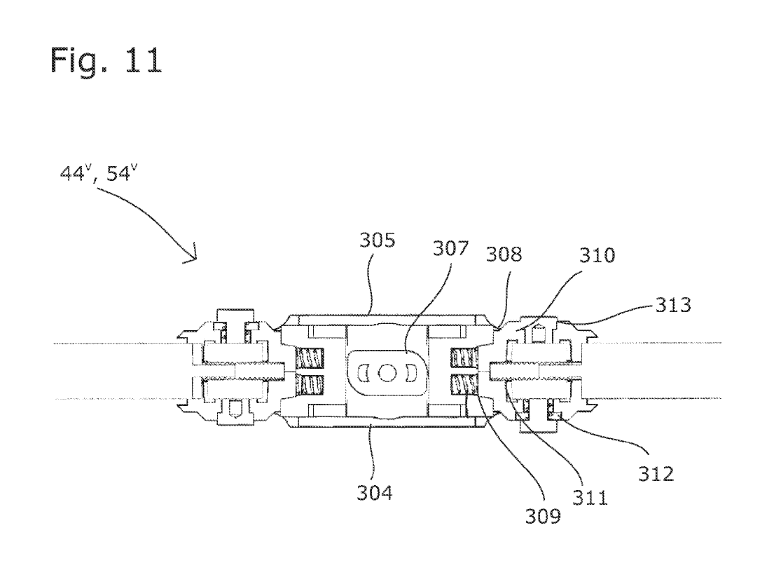

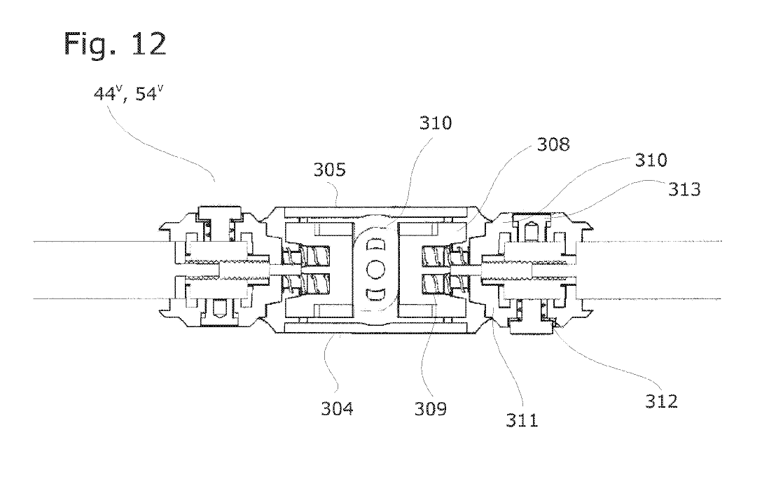

The FIGS. 10, 11, 12 show another embodiment of a chain link 44^{V}, 45^{V} of the main and/or side chain of the measuring device according to the invention in which locking means 306, 307, 308, 309 are provided additionally. The locking means are designed in such a way that, in the associated locking position, they lock the positive-engagement means or latching means 310, 311, which are also provided, in their engagement position. The joint fittings of this embodiment comprise latching means 310, 311, which are pressed onto each other by a screw connection 313 and a spring bias 312, and the associated furrowed surfaces come into engagement, as is shown in FIG. 11. The lock of the latching means 310, 311 is effected by means of a cam drive 306, 307. The latter is integrated into two half-shells 304, 305 substantially defining the chain link 44^{V}, 45^{V}. By adjusting the swivel axle 305, which is accessible from the outside and which is provided with a facility for applying a tool, a cam 307 is rotated on the inside, which respectively causes by means of its cam face a linear displacement of a U-shaped latching bar 308 against a spring bias 309. The latching bar 308 runs against a ramp-shaped portion of the latching means 310, 311 and urges their furrowed surfaces into engagement and with its arms finally grasps the latching means 311, 310 and locks them in their engagement position. Unlocking is effected by adjusting the swivel axle 306 and thus of the cam 307, which releases the latching bars 309, which are moved by the spring 309 into the unlocking position, as it is shown in FIG. 12.

What is claimed is:

1. Measuring device (1) for determining a surface contour (42) of a human or animal body (41), the measuring device (1) comprising a main chain (43) with a plurality of main-chain links (44, 44′, 44″, 44′″, 44^{IV}, 44^{V}) which are interconnected by a hinge joint (45), characterized in that the hinge joint (45) comprises a scale (13, 23, 74′) or sensors, with which the angle between the at least two main-chain links (44, 44′, 44″, 44′″, 44^{IV}, 44^{V}) can be read off or detected.

2. Measuring device (1) according to claim 1, characterized in that the measuring device (1) comprises at least one side chain (53) with a plurality of rotatably connected side-chain links (54, 54′, 54″, 54′″, 54^{IV}, 54^{V}), the side chain (53) being connected to one of the main-chain links (44, 44′, 44″, 44′″, 44^{IV}, 44^{V}) by means of a hinge joint (55).

3. Measuring device (1) according to the preceding claim, wherein the measuring device (1) comprises a plurality of side chains (53).

4. Measuring device according to any one of the two preceding claims, wherein the side chain is attached to a main-chain link (44, 44′, 44″, 44′″, 44^{IV}, 44^{V}) which respectively has two end portions (10) opposite along a first direction (100), each of which comprises a first joint fitting (11) for attachment to the further main-chain links, and the main-chain link (44, 44′, 44″, 44′″, 44^{IV}, 44^{V}) further comprises at least one second end portion (20) disposed at an angle to the first direction (100), which comprises a second joint fitting (21) for attachment of the side chain (53).

5. Measuring device (1) according to any one of the preceding claims, wherein the measuring device (1) comprises at least one means (200) for determining at least one direction of inclination of a main-chain link (44, 44′, 44″, 44′″, 44^{IV}, 44^{V}) or of a side-chain link (54, 54′, 54″, 54′″, 54^{IV}, 54^{V}).

6. Measuring device (1) according to the preceding claim, wherein the means (200) for determining at least one direction of inclination of a main-chain link (44, 44′, 44″, 44′″, 44^{IV}, 44^{V}) or of a side-chain link (54, 54′, 54″, 54′″, 54^{IV}, 54^{V}) comprises a bubble level (211) and a scale (213).

7. Measuring device (1) according to any one of the preceding claims, wherein a marker identifying the respective chain link, for example a numbering, is provided on each main-chain link (44, 44′, 44″, 44′″, 44^{IV}, 44^{V}) and/or side-chain link (54, 54′, 54″, 54′″, 54^{IV}, 54^{V}).

8. Measuring device (1) according to any one of the preceding claims, wherein the hinge joint (45, 55) comprises latching means, in particular positive-engagement means (14, 24, 34, 201, 203, 310, 311), for respectively latching two adjacent main-chain links (44, 44′, 44″, 44′″, 44^{IV}, 44^{V}), two adjacent side-chain links (54, 54′, 54″, 54′″, 54^{IV}, 54^{V}), and/or an adjacent main-chain and side-chain link (44, 44′, 44″, 44′″, 44^{IV}, 44^{V}, 54, 54′, 54″, 54′″, 54^{IV}, 54^{V}) in the respective angular position.

9. Measuring device (1) according to any one of the preceding claims, wherein the hinge joint (45, 55) comprises friction-engagement means (71, 72, 73) for respectively fixing by frictional engagement two adjacent main-chain links (44, 44′, 44″, 44′″, 44^{IV}, 44^{V}), two adjacent side-chain links (54, 54′, 54″, 54′″, 54^{IV}, 54^{V}), and/or an adjacent main-chain and side-chain link (44, 44′, 44″, 44 ′″, 44^{IV}, 44^{V}, 54, 54′, 54″, 54′″, 54^{IV}, 54^{V}) in the respective angular position.

10. Measuring device (1) according to any one of the preceding claims, wherein the hinge joint (45, 55) comprises locking means (306, 307, 308) for respectively fixing two adjacent main-chain links (44, 44′, 44″, 44′″, 44^{IV}, 44^{V}), two adjacent side-chain links (54, 54′, 54″, 54′″, 54^{IV}, 54^{V}), and/or an adjacent main-chain and side-chain link (44, 44′, 44″, 44′″, 44^{IV}, 44^{V}, 54, 54′, 54″, 54′″, 54^{IV}, 54^{V}) in the respective angular position.

11. Measuring device (1) according to any one of the preceding claims, wherein an adjustable longitudinal adjustment means (38) of at least one main-chain link (44, 44′, 44″, 44′″, 44^{IV}, 44^{V}) or side-chain link (54, 54′, 54″, 54′″, 54^{IV}, 54^{V}) is provided.

12. Measuring device (1) according to any one of the preceding claims, wherein the longitudinal adjustment means (38) comprises an elongated hole (37) and a further scale (33) disposed along the elongated hole (37).

13. Method for determining a surface contour of a human or animal body, in particular of a horse's back (41′), with a measuring device (1) according to any one of the preceding claims, wherein, in the first method step, the measuring device (1) is disposed on the surface (41′) and the measuring device (1) is adapted to the surface contour (42), and characterized in that, in a second method step, scale values or sensors of the measuring device (1) are read off or read out.

14. Method according to the preceding claim, wherein at least one direction of inclination of a main-chain or side-chain link, preferably of a main-chain link, is determined in the second method step.

15. Use of the measuring device (1) according to any one of the claims 1 to 12 for the manufacture, adaptation and/or checking of equestrian equipment, in particular a saddle or saddle rack.
